(19) United States
(12) Patent Application Publication
(10) Pub. No.: US 2016/0174840 A1
(43) Pub. Date: Jun. 23, 2016

(54) Physiological Monitoring Device and Related Methods

(71) Applicants: Emem Ufot Udoh, Maple Grove, MN (US); Tonya Sothan Heng, Maple Grove, MN (US); Trung Vu, Brooklyn Center, MN (US)

(72) Inventors: Emem Ufot Udoh, Maple Grove, MN (US); Tonya Sothan Heng, Maple Grove, MN (US); Trung Vu, Brooklyn Center, MN (US)

(21) Appl. No.: 14/839,987

(22) Filed: Aug. 30, 2015

Related U.S. Application Data
(63) Continuation-in-part of application No. 14/573,236, filed on Dec. 17, 2014.

(51) Int. Cl. A61B 5/00; A61B 5/11; A61B 5/145; A61B 5/04

(52) U.S. Cl. CPC A61B 5/0022; A61B 5/04; A61B 5/11; A61B 5/7475; A61B 5/7282; A61B 5/742; A61B 5/6831; A61B 5/4362; A61B 5/145; A61B 5/6898; A61B 2560/0475; A61B 2562/18; A61B 2560/0242

(57) ABSTRACT

Systems, devices and methods for monitoring the physiological parameters of a subject. The physiological monitoring device comprising (i) a physiological board assembled and enclosed in a material that conforms to a subject, (ii) a set of sensors disposed in the physiological board, (iii) a microcontroller unit, securely adapted to communicate to a network or cloud system. The network or cloud system includes a plethora of information associated with health conditions from multiple users, research studies, statistics and monitoring devices. The network or cloud system is operable to learn, analyze, store, display or provide useful feedback information to users to improve health conditions and experiences.

### CROSS-REFERENCE TO RELATED APPLICATIONS

The subject matter of the present invention is a continuation-in-part of U.S. patent application Ser. No. 14/573,236; entitled “Physiological Parameter Monitoring System” filed, Dec. 17, 2014. The above described application is hereby incorporated herein by reference in its entirety.

### BACKGROUND

The present invention relates to physiological parameters monitoring, more specifically to devices and related methods for monitoring the physiological conditions of a subject.

In a society that is getting increasingly conscious of health parameters and presented with more options to understand and study when, where and how to do more or less with the resources available regarding the health of the ones in our care, the use of thermometers, scopes and other equipments for such data is increasing. Society also reacts negatively when that collection of data is left with some measure of discomfort or is unusually intrusive. The health parameters of infants are especially important as they do not possess the capacity to voice out their ailments or bothers as do adults. Adults also prefer use of monitors that provide information that can be kept in the privacy of their reach without seeking the aid of third parties at every whim.

When there is need to find out how healthy someone is, preferably via vital signs, most people have to go to clinics, hospitals or similar facilities for tests and measurements. Sometimes, getting to such facilities depend on time of day or night, resources at the disposal of the intended person or even time to be allocated to such process. If the intended person is a baby, the rigor of taking the baby to a facility and time of such need create some inconvenience for the parties involved. The more information needed at each instance, the more complicated or engaged the inconvenience would typically be. As an example, if more parameters are needed to be monitored or checked, the more complicated the apparatus or setup it typically is for such information to be collected.

With the increasing need for monitoring human health and the everyday physiological activity, a robust, reliable, unobtrusive and comfortable way to acquire these physiological parameters is needed. The capacity to transfer or store such information is also an expectation. The devices and methods are provided in the present invention and claimed herein.

### SUMMARY

An object of the present invention is to provide a monitoring device or unit, which can be placed on a subject to precisely monitor the physiological parameters and issues an alarm upon sensing abnormal health conditions.

In some embodiments, the monitoring device or unit comprises a unit housing. The housing material is made of a synthetic, polymeric or fabric substance. The housing material consists of at least one of a water repellent or a water proof coating to protect the unit from moisture. Additionally, the unit housing will conventionally be of a thickness that allows one or more apertures of a predetermined dimension to be formed therein to support electronic components.

In some embodiments, the monitoring device or unit comprises a physiological board disposed with electronic components such as, one or more sensors, microcontroller unit, audio means, user interface, radio-frequency module or transceiver circuitry coupled to the board. The one or more sensors are electrically coupled to a microcontroller unit to measure electrical signals from a subject. The sensors may be a combination of sensors having at least one of a thermal, electrical, optical, mechanical, sound or chemical sensor. All sensors may be utilized to acquire various physiological parameters from the subject.

In some embodiments, the monitoring device or unit comprises a microcontroller unit to acquire one or more electrical signals from a tiered combination of sensors received from a subject, process in real time the electrical signals to determine at least one physiological parameters of the subject and generate alarm when adverse risk is detected. The microcontroller unit is designed to conserve energy by reducing the operational duty cycle and utilizing a predetermined signal processing window to allow monitoring of signals associated with the physiological conditions of the subject continuously for a period of time.

In some embodiments, the monitoring device or unit may comprise audio means. The audio means performs one or more operations to generate digital audio signal from an analog signal perceived as sound, or operations to generate an analog signal from a digital audio signal to output to a transducer. The circuitry performing these operations is configurable to at least decompress, decode or convert the signals into multiple formats of audio protocols.

In some embodiments, the monitoring device or unit may comprise an interface to communicate physiological conditions to users. The interface may be a display that is visual or may incorporate voice recognition, gesture, audio, touch screen, keypad, vibration or other interface technologies as recognized by those skilled in the art.

In some embodiments, the monitoring device or unit may comprise a transceiver circuitry or radio-frequency module for communicating to a network via a communication protocol. The transceiver circuitry is electrically coupled to the microcontroller unit for bidirectional communication, allowing monitoring of the physiological conditions of a subject from a network. The transceiver circuitry can be additionally configured to communicate the signals to a network by means of an intermediate device such as a base station. The microcontroller unit is adaptable to store data in a storage memory that specifies the communicating device identification to a network.

In some embodiments, in addition to the alarm being generated from the physiological monitoring unit, physiologic data will be communicated to networks or cloud systems and, possibly to third party(ies) such as caregivers via a communication protocol, thus allowing further alarms to be generated at the network or cloud system devices when readings are outside acceptable ranges.

In some embodiments, the present invention provides a configurable monitoring unit. The unit is operable to monitor physiological conditions continuously or in spot check. The unit may be configured to accept an external sensor by attaching the external sensor to a connector on the unit for processing and monitoring. The external sensor may include but not limited to sensors for blood glucose, hormonal changes, blood pressure, electrocardiogram, heart rate, oxygen saturation, among others. Preferably, the connector on the monitoring unit uses a configuration means that supports identifying and supplying power to the external sensor, which can be subsequently connected to or disconnected from the monitoring unit with more ease.

It is also an object of the present invention to provide related methods for monitoring physiological parameters of a subject.

An embodiment of the present invention provides a method for the monitoring unit, where active sensing and processing takes place in the unit.

An embodiment of the present invention provides method of monitoring where sensing and further processing are done remotely from a network or cloud system.

According to one embodiment includes a plurality of monitoring units, a plurality of user points or third party(ies), a plurality of network devices, a plurality of users and a network or cloud system. The network or cloud system comprises resources to learn, analyze determine, store or predict physiological events of a subject. The network or cloud system may further display a plurality of physiological parameters for multiple local subjects on a plurality of network devices. The local subject monitoring unit may display a context from the network or cloud system on its user interface. The context display may include a plurality of data, respectively representing health conditions from a plurality of users. The plurality of network devices, plurality of monitoring units and plurality of user points or third party(ies) devices can be accessible from the network or cloud system. The monitoring units, network devices, user points and the network or cloud system are configured to at least send and receive data packets via a communication protocol. The data packets may contain electrical, physiologic or audio data.

The monitoring unit of the present invention may be adaptable for use with a strap or wrap to access the physiological characteristics available at a tissue level, thereby allowing monitoring of the health and activity level of a subject. The measurement acquired by the monitoring unit provides a snapshot of a subject's condition taken at a particular time as these parameters can change very quickly or may even go undetected in the absence of monitoring.

The systems, devices and methods described herein may be deemed applicable to other settings. Depending on need and application, embodiments of the present invention can be used in several situations such as clinical, home, foster care, nursing homes, day care facilities, group homes or other desired settings. For a better understanding of the objectives, features and advantages of the present invention, references should be made to the following detailed description and accompanying drawings.

### BRIEF DESCRIPTION OF THE DRAWINGS

FIG. 1 shows an exploded view of an embodiment of the physiological monitoring unit according to the present invention.

FIG. 2 is a schematic view of an embodiment of the physiological monitoring system according to the present invention showing the monitor and communication system.

FIG. 3 is another schematic view of an embodiment of the physiological monitoring system according to the present invention showing the monitor and communication system.

FIG. 4 is another schematic view of an embodiment of the physiological monitoring system according to the present invention showing aspects of the monitoring unit.

FIGS. 5A-5B illustrates related methods for monitoring physiological parameters of a subject according to the present invention.

FIG. 6 is another schematic view of an embodiment of the physiological monitoring system according to the present invention showing the monitor wrapped around the stomach of an expectant mother, wrist or arm of a user and communication system.

FIG. 7 is another schematic view of an embodiment of the physiological monitoring system according to the present invention showing the monitor wrapped around the stomach of an expectant mother, wrist or arm of a user and communication system.

FIG. 8A is a view of an embodiment of a monitor usable in the present invention showing a removable port and an extended adapter.

FIG. 8B is a side perspective view of an embodiment of a monitor usable in the present invention.

FIG. 8C is another perspective view of an embodiment of a monitor usable in the present invention showing a removable port and an extended adapter.

FIGS. 8D-8I are different views of a monitor usable in the present invention.

FIGS. 9A-9B are side perspective views of an embodiment showing an exemplary monitor usable in the present invention.

### DETAILED DESCRIPTION

The following description of the invention references the accompanying drawings that illustrate specific embodiments in which the invention may be practiced. In accordance with each preferred embodiment, the invention provides system, device and related methods for monitoring physiological parameters of a subject. Other embodiments may be utilized and changes may be made without departing from the scope of the invention. The following description is, therefore, not to be limited, as various modifications or changes may be made by those skilled in the art.

In this description, reference is made to a pending U.S. patent application entitled “Physiological Parameters Monitoring System” with application Ser. No. 14/573,236. Applicant further makes references to “embodiment” or “embodiments” to mean that the feature or features being referred to are included in at least one embodiment of the present invention and do not necessarily refer to the same embodiment and is also not mutually exclusive. Thus, a more complete understanding will be realized in view of the following embodiments with reference to the drawings and claims therein.

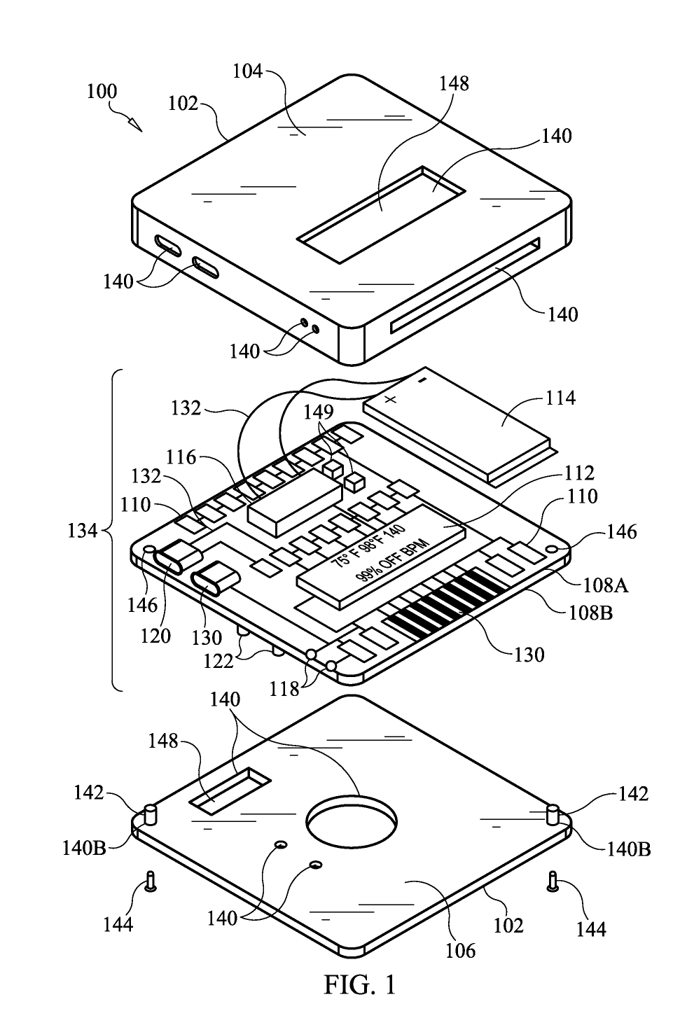

FIG. 1 shows an exploded view of a monitoring unit 100 according to an embodiment to the invention. The monitoring unit 100 includes a unit housing 102; comprising a top portion 104 and a bottom portion 106, a first physiological board 108A, a second physiological board 108B. The physiological board 108 comprises one or more electronic components 110 such as a set of sensors, a microcontroller unit, circuitry, a radio-frequency module, an interface 112, a battery 114, connectors 116, adaptors 130, indicators 118, a battery stand 149, a button 120, electrically connected and mounted on the board. Although, the physiological board is disclosed with reference to its preferred embodiments, the invention should not be limited thereby. Alternatives may be employed, for instance, the physiological board may be a flex or rigid printed circuit board or may include any combination of the same or the like. In some embodiments, the physiological board is positioned over the battery with connector 116 or other fixed form components that are terminally coupled to receive communication and/or connection via cables, wires 132 or via adaptors 130. The adaptors snap to the board comfortably and easily. In some embodiments, the physiological board may comprise a segmented single, double or multilayered disposition of electronics and other components. In some embodiments, the physiological board may be encased in the unit housing 102, and the unit housing coated with a conductive water resistant material capable of electrically coupling to a subject. In some embodiments, the physiological board disposed with electronic components 110 and battery 114 may be coated together 134 with a conductive water proof material to inhibit water flow into the monitoring unit. In some embodiments, the physiological board may comprise a first, second or more physiological boards for various design considerations and application needs.

The unit housing 102 positions the physiological board 108 and a plurality of electronic components 110 with apertures 140, allowing one or more sensors 122 to protrude a predetermined distance from the unit housing surface to be in contact with a subject. In some embodiments, the bottom portion 106 of the unit housing 102 includes apertures 140B comprising a positioning member 142 in the form of mounted structures to allow fasteners 144 to hold the physiological board via a recess 146 in firm position, thus preventing unwanted movements of the sensors on an application site during monitoring. In some embodiments, the unit housing includes apertures 140 with a conductive covering 148, the conductive covering made from an opaque material to reduce interference from an external light source is positioned to provide electrical coupling between the protruding sensors and the subject. In some embodiments, the unit housing 102 may comprise many known pliable materials such as silicone or the like that has no adverse risk to the skin of the wearer, water resistant or water proof material that can impede the flow of moisture or water into the monitoring unit. The apertures are advantageous for skin contact allowing sensors to noninvasively transmit and receive acoustic or optical impulses. In furtherance, the aperture spacing and diameter may be adjusted to suit different application needs.

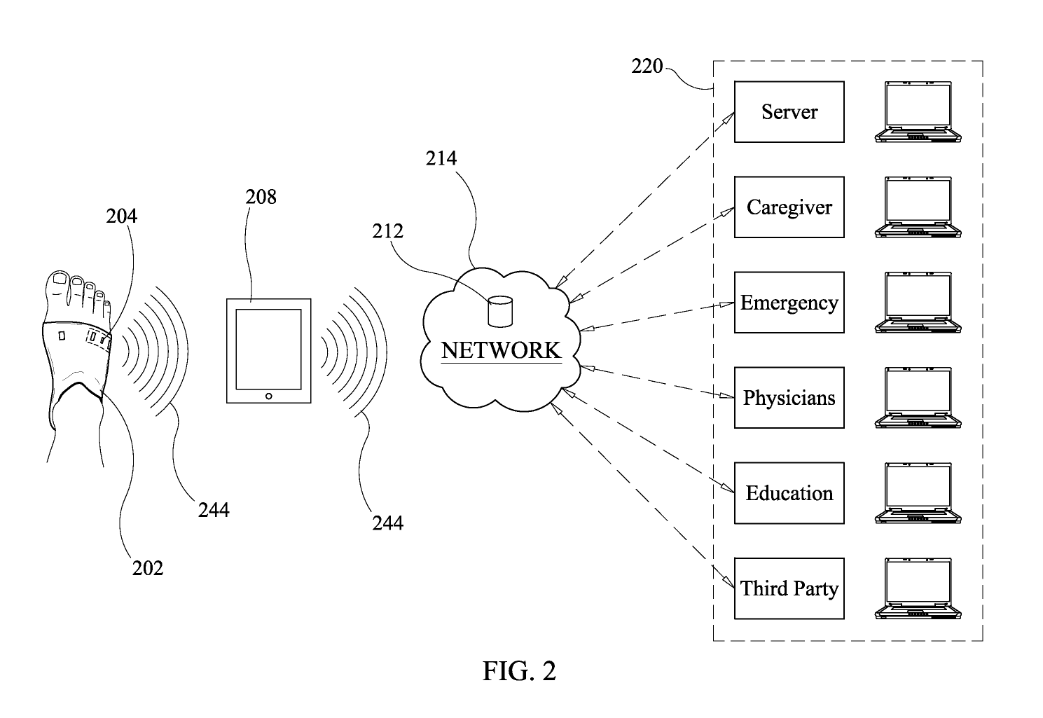

According to the present invention, FIG. 2 preferably illustrates an information collection system with sensors or sensory units 204, a transmitting means 244 for communicating with a base station 208, a further transmitting means 244 for communication with a network or cloud system 214, wherein said network or cloud system comprising a database 212 and a plethora of user points 220 for servers, caregivers, emergency services, physicians, educators or third party. The transmitting means may be wireless or wired (not shown), adaptable to be used to obtain the needed information for the monitoring of a subject according to the present invention. In some instances, wireless or wired transmission of the data using a base station 208 may include an enhancement to assure data connectivity with the network base or collecting station. It is reasonable to include a power supply unit, booster or charging mechanism to provide such enhancement for the assured data transfer between locations according to the present invention. As indicated, base station 208 preferably includes a mobile connectivity as is known in the art for use in sharing or transferring data to a network system 214 that may or may not include use of a database 212. Data processing or management may preferably take place within the network 214 or database 212 in concert with users and outlets such as servers, caregivers, emergency services, physicians, educators and other third parties, collectively 220. Those skilled in the art are typically conversant with such systems and the application of such services to the management and use of the data or information collected.

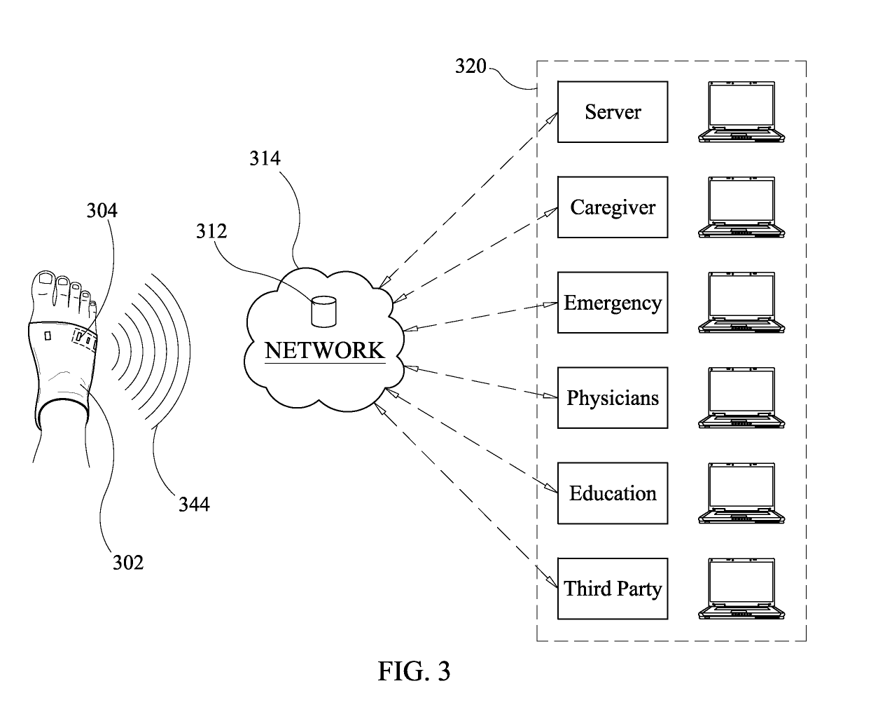

In yet another embodiment of the present invention and in the manner described above, FIG. 3 preferably illustrates an information collection system with sensors or sensory units 304, a transmitting means 344 for communication with a network or cloud system 314, wherein said network or cloud system comprising a database 312 and a plethora of user points 320 for servers, caregivers, emergency services, physicians, educators or third party. In this embodiment, the data or information collected is directly communicated or transferred to the network or database without an intervening step or process of communication with a base station. As disclosed above, the communication system of the instant invention may be wireless or wired depending on application and circumstance of use.

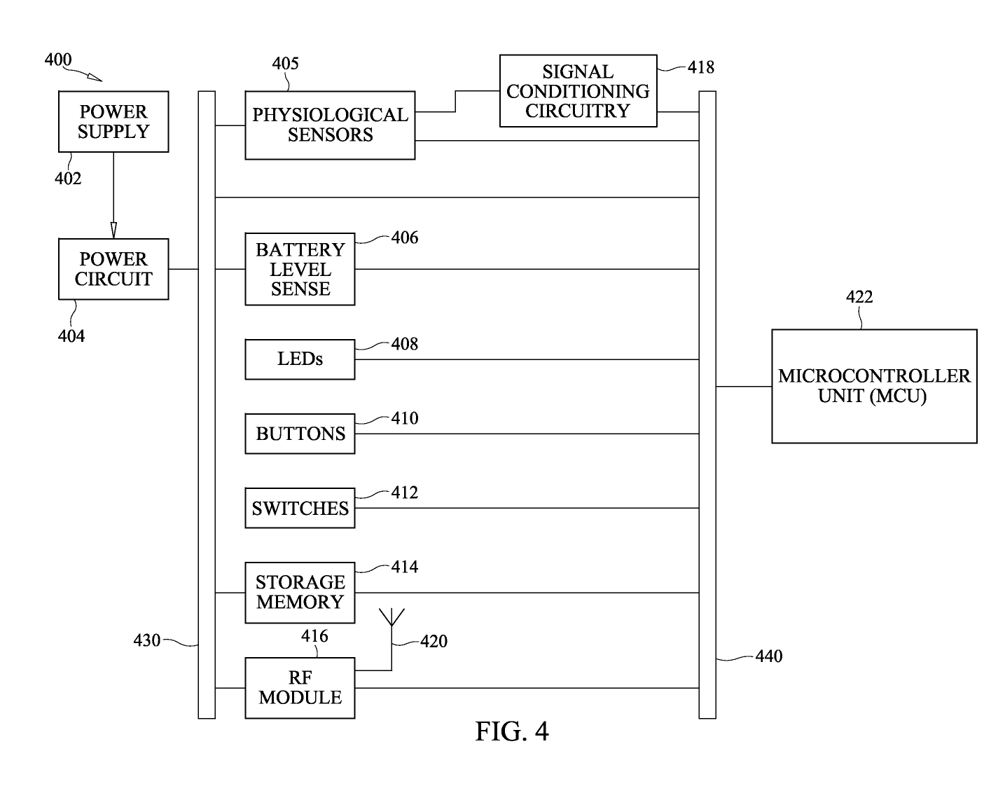

FIG. 4 illustrates a block schematic diagram of a preferred embodiment of the monitoring unit according to the present invention 400 illustratively showing a power supply 402 linked to a power circuit 404 including a power bus 430, a set of physiological sensors 405, signal conditioning circuitry 418, a battery or power level sensor 406, light emitting diodes 408, user managed buttons or keys 410, user managed switches 412, a storage memory 414, a radio-frequency module 416, an antenna 420, a communication bus 440, and at least a microcontroller unit 422. The physiological sensors can and may be very broad to include most advances in medical science and technology to non-invasively collect pertinent data for the assessment of the user's condition. Such sensors include but are not limited to electro-cardiogram (ECG), motion, movement, orientation, accelerometer, heart rate, blood glucose, respiration rate, temperature, weight, blood pressure, activity level, switches, indicators and other buttons. A signal conditioning circuitry may be incorporated for use in filtering, amplifying and/or isolating the output or input into the physiological sensors. The RF module 416 of the present invention may be wired or wireless for use as cellular, Wireless Fidelity (WiFi), Bluetooth, Ethernet, or other systems and protocol as known to those skilled in the art. The power supply 402 according to the present invention may preferably be wireless or battery powered.

The microcontroller unit 422 comprise methods to judge physiological parameters related to fertile period, antepartal, fetal care, neonatal, infant care and adults; digital filters and windows for signal conditioning; look-up table derived from various physiological characteristics of healthy subjects at various time; curves to calibrate sensors and other electronic components; resources for memory constraint, energy saving, signal processing, analysis and data storage; software pre-processors for the unit or device firmware and configuration means for external sensors or adaptors; cryptography mechanism and other security measures; users and device communication protocol stacks (i.e., TCP/IP, Bluetooth Stack); among others. The radio-frequency module 416 is coupled to the microcontroller unit and can be utilized to upgrade the related methods using different communication standards such as over-the-air-programming. All the modules, blocks and circuitry of the monitoring unit are powered by the power bus 430, and inter-circuitry communication via the bus 440.

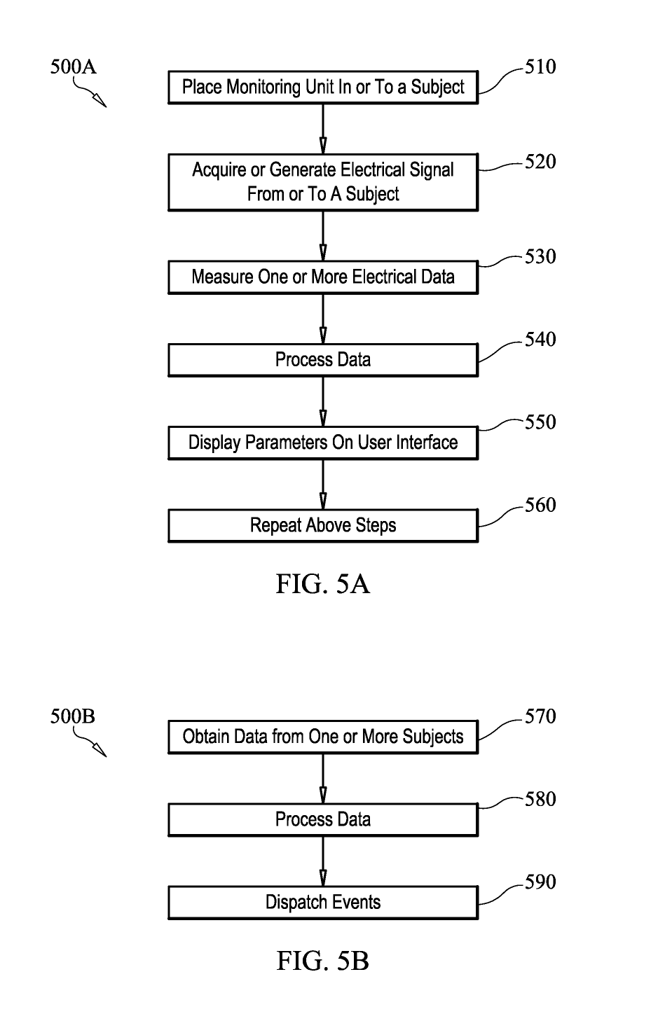

FIGS. 5A-5B illustrates related methods (500A and 500B) for monitoring physiological conditions of a subject according to the present invention. Other sequences of steps may be performed according to alternative embodiments, which may not be limited to include going through the steps outline in a different order, combining multiple sub-steps in various sequences as needed for other applications. Additional steps may be added or removed depending on the particular need as recognized to those skilled in the art.

In step 510, a monitoring unit (i.e., 204, 304, 604, 704) is placed to interact with a subject in order to derive a time-varying electrical measurable signal. In an example, the monitoring unit 604 is placed on the wrist or arm of a subject 602, appendage of subject 202, stomach or belly of subject 702 or positioned substantially toward a fetus 706. In an embodiment, the monitoring unit is placed on the skin of a subject (i.e., 202, 302, 602, 702). Other skin application site may include the finger or chest of the subject. In certain embodiments, step 510 attaches the monitoring unit to a wearable strap 610 or wrap 604, or electrically connects the monitoring unit to an external sensor or adaptor 832, and places such combination on the skin of the subject or in a bodily fluid such as blood, urine, sweat or tear. In some embodiments, the monitoring unit may be utilized in an environment to measure ambient temperature or humidity in the atmosphere. In some embodiments, the monitoring unit may be in communication with a network or cloud system (i.e., 214, 314, 614, 714) in this step to receive configurable data commands for a particular monitoring mode of operation.

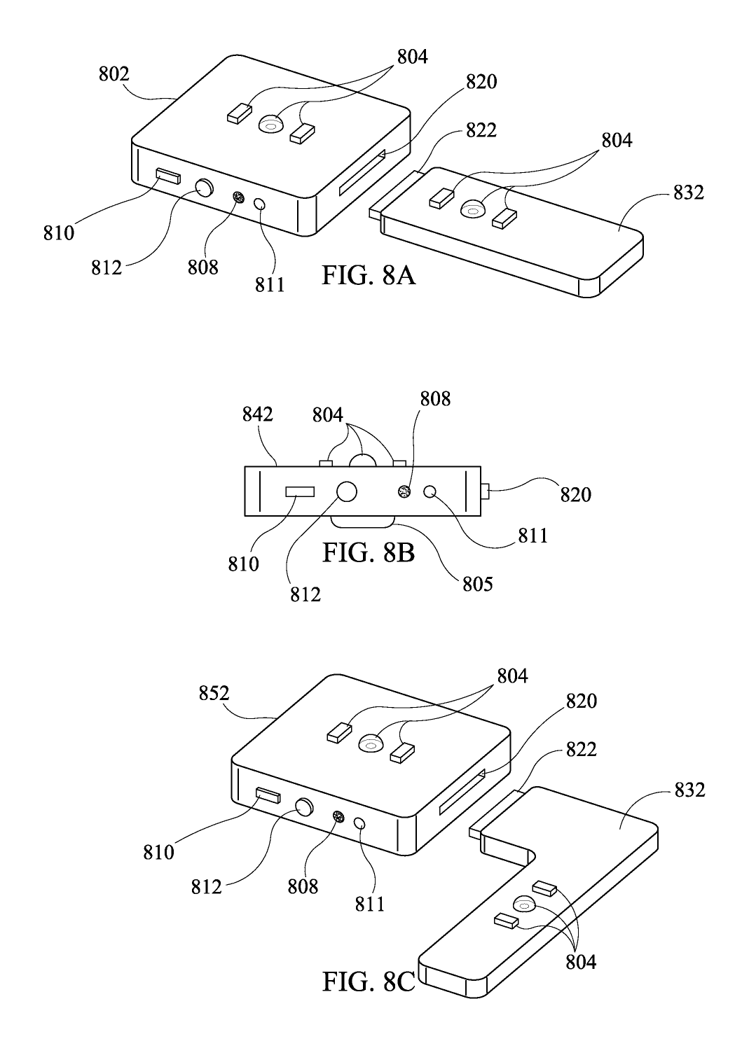

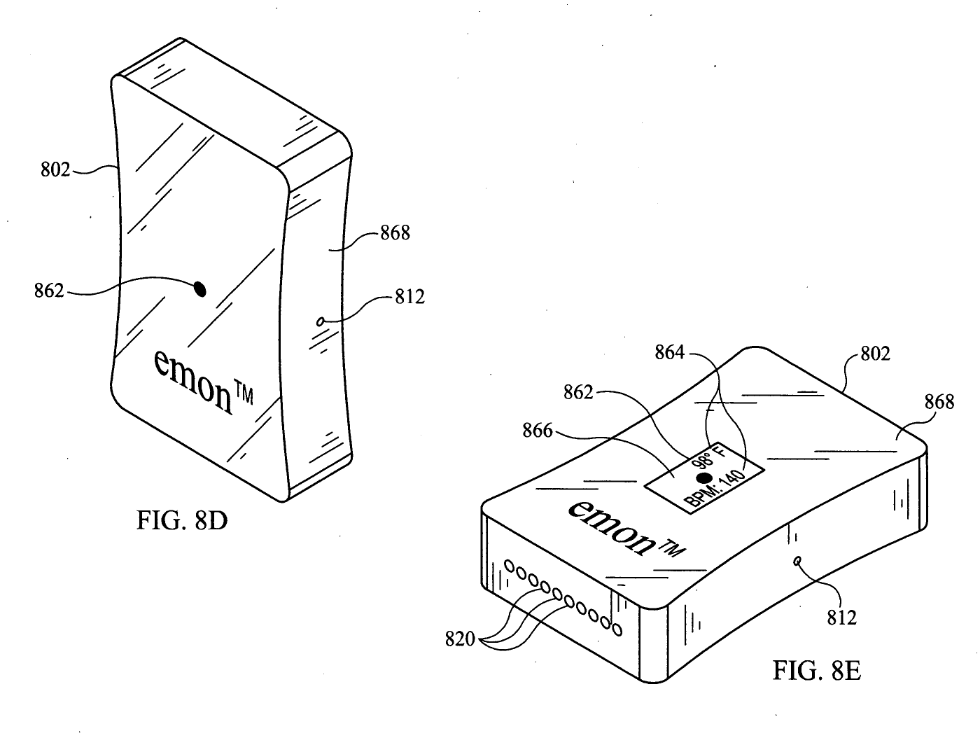

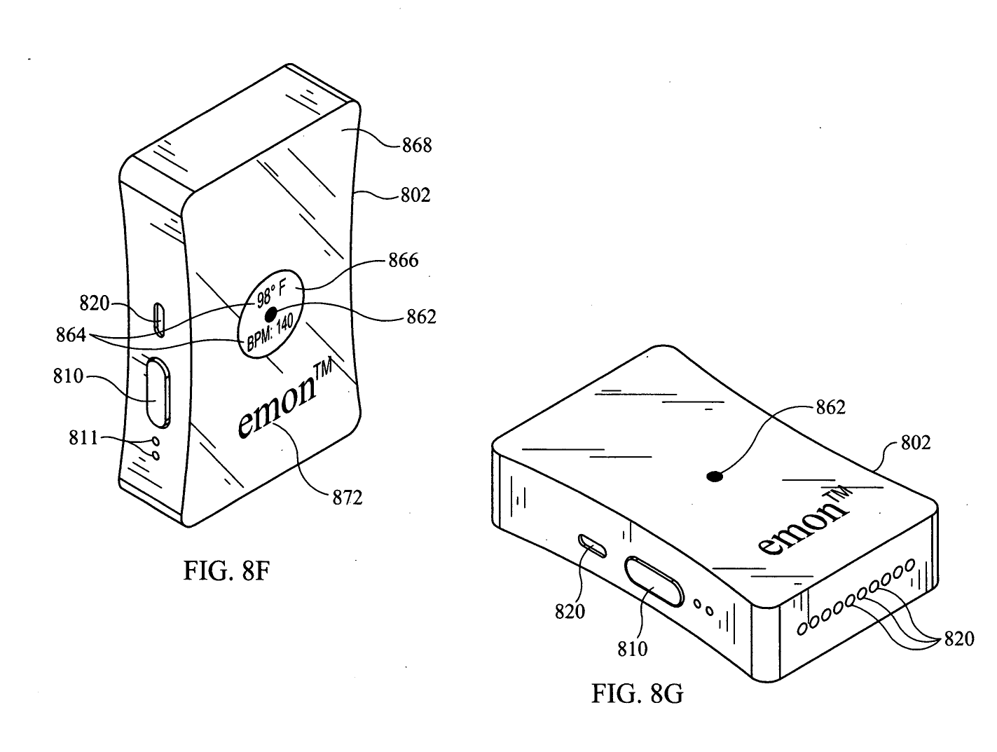

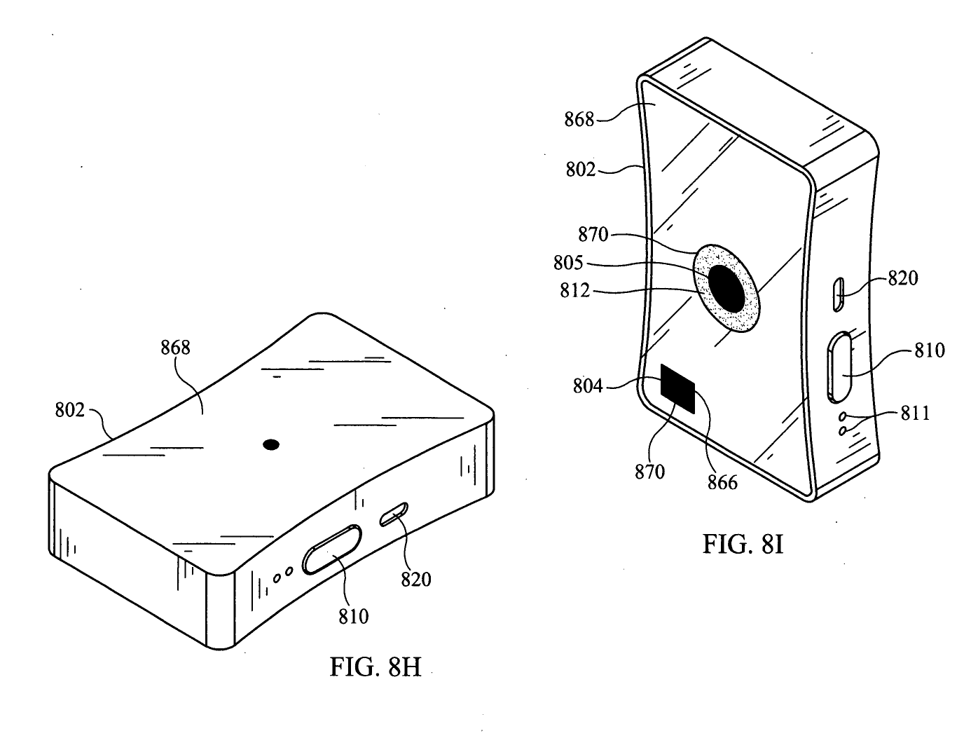

In step 520, a monitoring unit (i.e., 204, 304, 604, 704, 802) acquires time-varying electrical signals from a subject (i.e., human skin, bodily fluid or atmosphere) via one or more sensors 405 coupled to a microcontroller unit 422 (FIG. 4) or from an external sensor 832 (FIG. 8) attach to the monitoring unit via a connector 820. For example, the sensor 805 in unit 802 includes sensors configured to sense heart rate, movement and other physiological conditions of a fetus 606 in a stomach or belly of the subject 602. In certain embodiments, the monitoring unit is configured to generate signals to the subject and measure the following reflected signals generated by the subject via one or more sensors 405. For example, an optical sensor with first light emitting diode 804 emits light rays and a second light sensing diode 804 measures the reflected or following signal. In other example, acoustic ultrasonic sensor 805 emits sound pulse and measures the time-varying reflected signals from the tissue to detect breathing in a fetus.

In step 530, the electrical signals obtained are quantified into first, second or more sets of physiologic data based on at least a plurality of measurement of said electrical signals and/or external input data if requested and received that can be processed in 540. In step 540, the physiologic data or signals are processed by the microcontroller unit 422 (FIG. 4) of the monitoring unit 400 (FIG. 4) to determine one or more physiological conditions. For example, the monitoring unit is configured to generate an analog signal from a digital signal to output to a transducer such as ultrasounds, headsets, speakers, or generate a digital signal from a analog source perceived as sound or from a transducer such as microphone at 418, read and store data in a storage memory 414, determine whether to request an external input data from an external sensor 832, an user interface 862, a button 810, or a network or cloud system (i.e., 214, 314, 614). The external input or data may be a measurement selected from blood glucose, iron level, blood pressure, electrocardiogram or personal information. In some embodiments, the monitoring unit may write measurable data, physiologic data, one or more conditions to be transmitted to a base station or cloud system (FIGS. 2, 3, 6, 7) via a communication means (i.e., 244, 344, 644, 744) for further processing. Other processing may involve learning, predicting, determining a disease state of a subject and generating alarm when adverse risk is detected.

In step 550, the measurable data, physiologic data, one or more conditions are display on a user interface (i.e., 114, 862) or as light emitting diode indicators (i.e., 811) to users (i.e., 202, 302, 602, 702) on the monitoring unit. The user interface receives input or data from users and visually display physiological parameters, health conditions, status indicators, diagnostic data, output information, in the form of a screen such as liquid crystal display (LCD) or through an array of illuminated light such as light emitting diode (LED) during operation. The user interface may incorporate audio (i.e., 812) such as voice recognition, vibration or other user interface technologies as recognized by those skilled in the art. Continuous monitoring of a subject for a period of time in step 560 can be attained by repeating the above mentioned steps for abnormal or adverse risk detection.

In yet another embodiment, FIG. 5B shows a method 500B of monitoring subjects. The method includes a step 570 of obtaining a plurality of data from one or more subjects such as a plurality of users (i.e., 202, 302, 602, 702), a plurality of monitoring units (i.e., 204, 304, 604, 704), a plurality of network devices (i.e., 208, 608), a plurality of network or cloud systems (i.e., 214, 314, 614, 714) and a plethora of user points (i.e., 220, 320, 620, 720) via a communication means (i.e., 244, 344, 644,744). The plurality of data received is process in step 580 to determine physiological states or conditions of the subjects, and generate intelligent alarms, alerts, notifications, results to be dispatched in step 590 to caregivers, emergency, physicians or third party(ies) devices having a communication link. This step may involve displaying data on a base station's user interface (i.e., 208, 608) or communicating said data to the monitoring unit user interface (i.e., 144, 862) via communication means (i.e., 244, 344, 644, 744) or said data made accessible from a plethora of user points.

The data displayed or transmitted may include but are not limited to feedback information about a user health conditions, intervention to abnormal physiological changes, indication that a disease state is present or reward output to encourage a user to be healthy. The physiological states or conditions are activity from a user or multiple users, related health topics pertaining to a subject or multiple subjects, conditions such as syndromes, disorders, symptoms, dysfunctions or disease detection, response to related concerns and issues, relevant information to know, to do and to expect about health, statistics aggregated from multiple health sources, apgar score and history of a subject or multiple subjects. In some embodiments, the data, the physiological states or conditions, alarms, alerts, notifications, results are received from users or subjects by means of the user points, the base station's user interface, and the monitoring unit user interface via the communication means in this step.

The related methods allows continuous monitoring of the physiological parameters associated with fertility, activities of daily living, pregnancy or prenatal. Combining the use of sensors with wireless communication technology enables remote monitoring and periodic feedback such as alerting caregivers or patients of potential warnings and abnormal physiological changes while they are away from clinical care settings. The automatic collection of pertinent data will be necessary to predict fertility, detect pregnancy, monitor prenatal ailments and determine the efficacy of administered treatments over an extended period of time. Hence, allowing caregiver or women to intervene before an ailment become irreversible. This is necessary to prevent fetal mortality, morbidity and unplanned pregnancy.

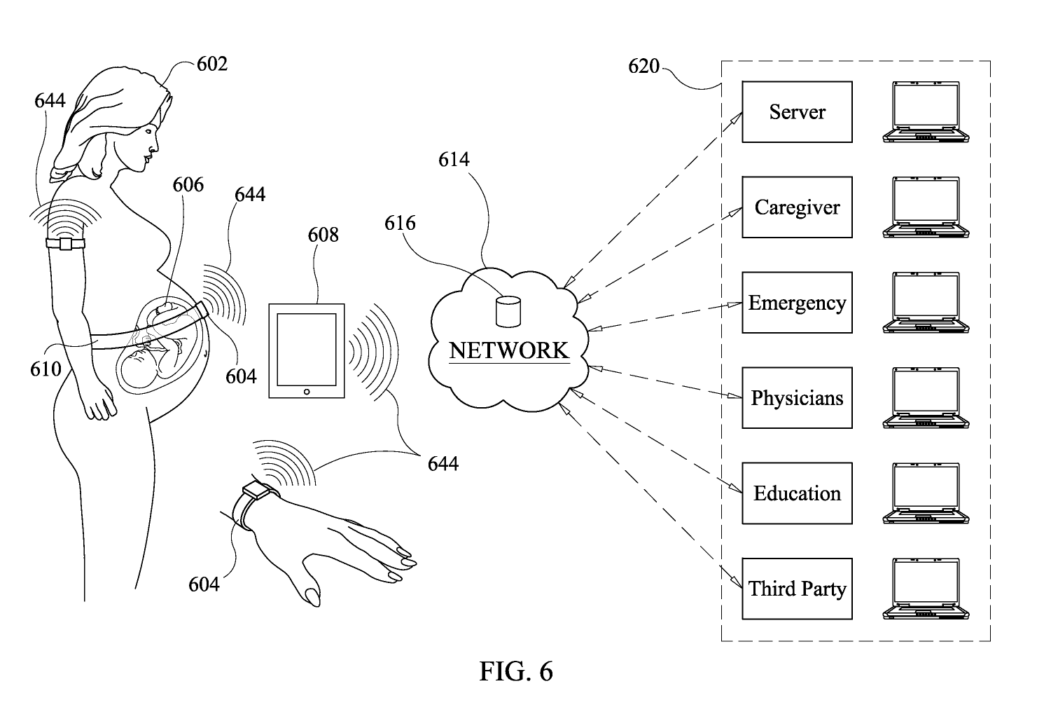

Now referring to FIG. 6, an expectant mother shown in 602, with a monitoring unit on the arm, wrist or belly 604, a fetus 606, a transmitting means 644, a base station 608, another transmitting unit 644, a network or cloud 614 with a database 616, in communication with users 620 that may include servers, caregivers, emergency service providers, physicians, educators or other third party(ies). As discussed above, the physiological sensing or monitoring unit 604 may be worn around any potential source of information to assess the physiological status of the fetus or object of interest. A list of prenatal or fetal monitoring configurations may be formed or established using the equipment and formations according to the present invention. The monitoring units 604 when used by a woman may be configured to provide physiological information suitable to indicate fertility opportunities such as peak ovulation times for the woman.

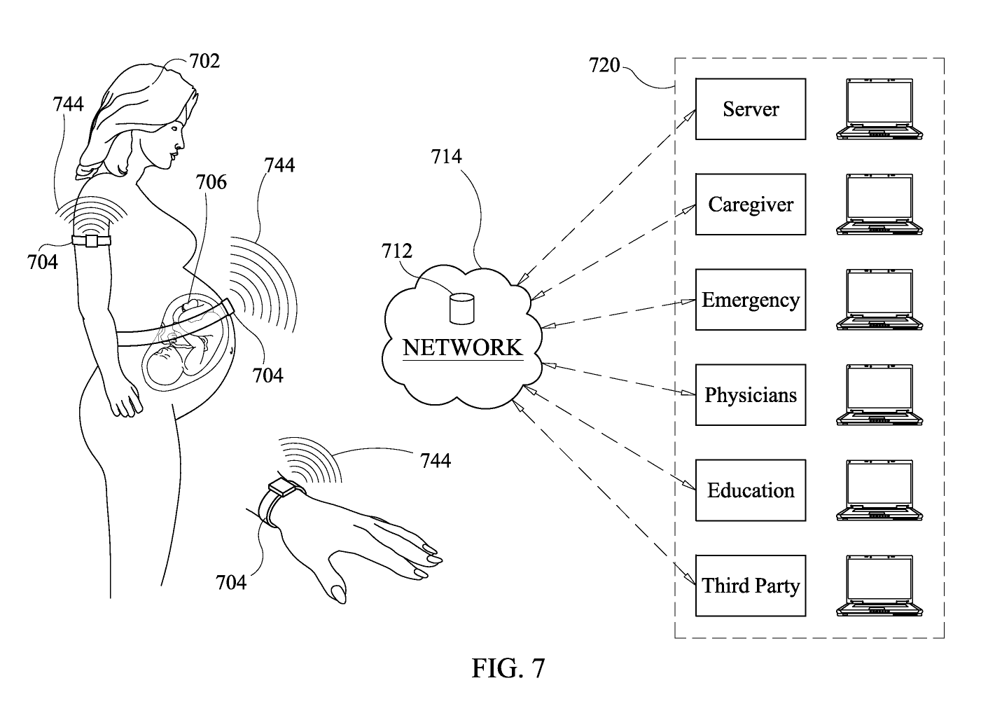

In yet another embodiment and in furtherance of the discussion above, shown in FIG. 7, an expectant mother shown in 702, with a monitoring unit on the arm, or wrist or belly 704, a fetus 706, a transmitting unit 744, a network or cloud 714 with a database 712, in communication with users 720 that may include servers, caregivers, emergency service providers, physicians, educators or other third party(ies). According to the present embodiment, data or information collected may preferably be directly communicated or transferred via a transmitting means 744 to a network 714 or database 712 without the need for an intervening step or process of communication with a base station 708. As discussed above, the physiological sensing or monitoring unit 704 may be worn around any potential source of information to assess the physiological status of the fetus or object of interest. A list of prenatal or fetal monitoring configurations may be formed or established using the equipment and formations according to the present invention. The monitoring units 704 when used by a woman may be configured to provide physiological information suitable to indicate fertility opportunities such as peak ovulation times for the woman.

Referring back to FIGS. 2, 3, 6 and 7 shows a system for monitoring the physiological parameters of a subject. The system includes multiple monitoring units (i.e., 204, 304, 604, 704), a plurality of network devices (i.e., 208, 608), a plurality of users (i.e., 202, 302, 602, 702), a plethora of user points or third party(ies) devices (i.e., 220, 320, 620, 720) along with one or more network or cloud systems (i.e., 214, 314, 614, 714). In operation, the monitoring units acquire electrical signals from a subject via one or more sensors. The units can be deployed at different application sites from which signals can be collected to monitor physiological characteristics. For example, wrist (FIG. 6), foot (FIG. 2), arm, or belly (FIG. 7). The physiological characteristics may be display on the monitoring unit human interface or transmitted via communication means (i.e., 244, 344, 644, 744) to a network or cloud system with a unique device identification for further processing. The network or cloud system, depending on request may learn using advances in artificial intelligence, analyze data from a plurality of subjects, store weighted combination of data received from multiple users, subjects and sources, or display aggregated health information from a plurality of users on its computing devices user interface. The network or cloud system is adaptable to monitor various kinds of conditions such as fertility, pregnancy, antenatal, neonatal, infants, fetal, gravida, among others, from multiple users continuously for a period of time.

The base station (i.e., 208, 608) may conventionally be a computing device such as personal digital assistants (PDAs), smartphones, tablets, computers, laptops, workstations, cameras, watches or other devices with communication link. The user interface of the monitoring unit, user point, and base station will provide a dashboard overview and act as a visual means for users to view personalized physiological characteristics and other forms of information. The user interface provides an interactive means to request, receive, and display data. The physiological characteristics and information (i.e., activity level, health data, statistics) can be displayed in different units, time (i.e., daily, weekly, monthly, yearly). The activity level comparison between multiple users or subjects being monitoring may be presented to access health experience. All data measured or received from users or subjects will require authentication in a manner that support privacy and protection.

With the use of a network or cloud system, physiologic and other data received from a plurality of monitoring units, base stations and users can be made accessible to a server, caregiver, emergency units, physicians and others. It is reasonable to include research and other users such as parents, as users of information available from the systems, devices and methods of the present invention. The transmission of data between the monitoring unit, the base station and the network or cloud system can be done wirelessly or wired via Antenna, Bluetooth, Wireless-Fidelity (Wi-Fi), Zigbee, Voice-Over Internet Protocol (VOIP), Cellular Network, Infrared, Wi-Max, Optocouplers, Near-Field Communication (NFC), Multi-Hop Communication, ANT, Universal Serial Bus (USB), Firewire, Serial Bus, Mobile-to-Mobile (M2M), or other now or later known communication processes. The data transmission may be adapted to use cryptography to securely transfer data, thus preventing unauthorized access.

In FIGS. 8A-8C perspectives of a monitoring unit 802 showing light emitting and sensing sensors or diodes 804, 842, 852, audio means 812, acoustic ultrasonic sensor 805, buttons 810, temperature sensors, battery level sensors and more for use in monitoring the physiological parameters of interest according to the present invention. Also included is a detachable connector 832 having some additional or duplicated sensors 804 and connector 822. Said connector 822 may be usable as a charging mechanism for the monitoring unit and is preferably adaptable to connect with the monitoring unit at 820. Said audio means 812 having hardware circuitry to detect, play and record sound signals.

FIGS. 8D-8I present perspectives of an exemplary assembled monitoring unit 802 having a user interface 862 permitting users to read information from a display means 864 visible through a conductive covering 866 which is recessed inside a unit housing 868. The unit housing 868 of the monitoring unit 802 is generally made of a rigid or flexible, synthetic, polymeric, fabric material to enclose and protect the physiological board, battery, electronic components 804, 842, 852, 812, 805, 810, and other circuitry from moisture or forces. The unit housing is joined by a wristband, armband, foot wrap or stomach strap system as shown in FIGS. 2-3, FIGS. 6-7 and FIGS. 9A-B which operates to create a comfortably wearable system that easily adheres to the skin to monitor physiological parameters of a wearer. The preferable material for use in the present invention should be soft, pliable and of such quality to not irritate or otherwise cause adverse condition to the skin of the user. Typical materials such as medically acceptable conductive material that would not store heat or cause heat to be generated at the application site. Also included is a detachable external sensor 832 having additional sensors 804 capable of electrically coupling to a subject and configurable to communicate with the monitoring unit at 820 via connector 822.

The additional sensors 804 may include chemical sensors for bodily fluids such as hormonal changes, drugs, solids, granules, enzymes, impedance, bioelectrical resistivity, tissue resistance, antibody or substance that may be detected in body fluids that may serve as a sign of a disease or other abnormalities; thermal sensors such as thermistor, thermocouple, or infrared, to measure the operating temperature of the monitoring unit, the ambient temperature of the air surrounding the subject or body temperature of the subject; sound sensors such as ultrasound to measure the amount of fluids in a subject's lungs; motion sensors configured to sense fetus movements, motions, kicks and positions in the belly of a subject; MEMS sensors such as accelerometer, gyroscope, magnetometers; optical sensors for heart rate, blood oxygen; electrical sensors for ECG; mechanical sensors for blood pressure; among others for monitoring.

The monitoring unit 802 may additionally provide electronic and configuration means for its inner circuit operation such as audio means 812, user interface 862, electronic circuitry and others components to be manipulated and operated from tactile devices such as key, keypad, button 810, or connector 822. Such electronic and configuration means may be setting the monitoring unit to monitor physiological parameters continuously or in spot check mode, or to accept, identify, configure or power an external sensor by allowing the external sensor to be attach to a connector on the monitoring unit for further processing. The unit housing 868 positions the tactile components proximately to one another such that each component protrudes from a recess 870 to remain in contact with the subject, or as an indicator of the monitoring unit operation made visibly to users via light emitting diodes 811, 808.

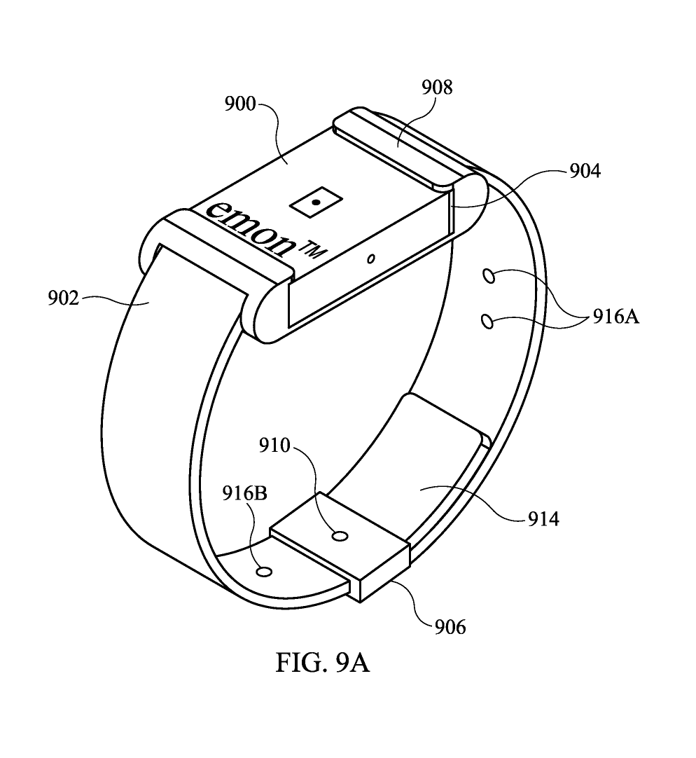

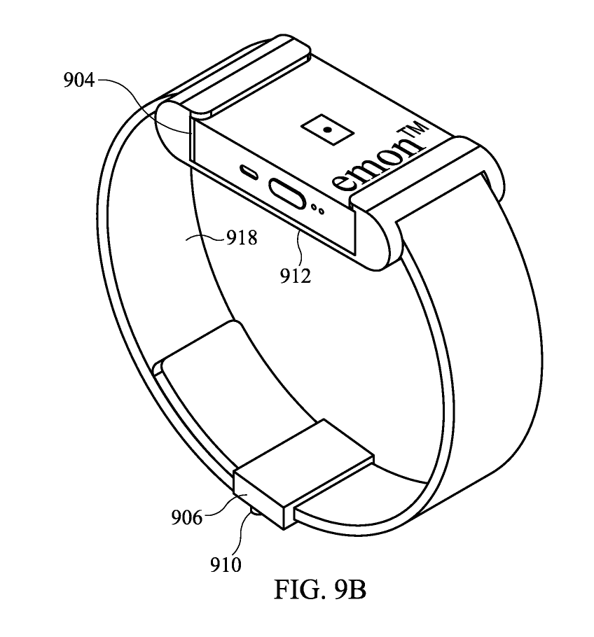

FIGS. 9A and 9B illustrate an attachment method comprising a strap or wrap 902 for placing the monitoring unit 900 on a selected application site, according to an embodiment of the invention. The strap or wrap 902 includes an adjustment assembly 906, an adaptor assembly 904, a pressure applicator 908, a button hole style 910, a conductive material 912, a positioning member 914, and sensors 916 (A, B). The method adheres the monitoring unit 900 to the wrap 902 to obtain physiological features via the conductive material 912 that remains in contact with the skin of a subject. The adjustment assembly 906 enables the wrap 902 to form fit around the application site. According to some embodiments, the wrap 902 comprises a pliable material such as fabric, any suitably stretchable or breathable material adapted to allow flexibility with body movements when the monitoring unit is placed on the subject. Additional pressure applicator 908 is included to apply and focus pressure on the monitoring unit 900 to be in firm contact with the adaptor assembly 904 and conductive material 912 when applied to the skin of the subject. In some embodiments, the pressure applicator 908 may comprise a soft flexible material that has no adverse risk to the skin of the subject. In some embodiments, one or more sensors 916 are embedded inside the strap or wrap 902 for additional sensing capability. The button hole style 910 is configured to receive the positioning member 914 to adjustably encompass the application site (i.e., the subject's wrist, arm, stomach or foot).

The use of the demonstrated embodiments is not desired a limitation of the scope of the invention. Those of skill in the art will understand and recognize that a variety of designs, modifications, adaptations, omissions, combinations, and other changes are practical and plausible, and all such variations are within the scope of the present invention, as set forth in the following claims.

What is claimed:

1. A monitoring unit, comprising:
a first sensor, said first sensor is at least one detachable external sensor having a first connector to attach to a monitoring device and a one or more sensors capable of electrically coupling to a subject to generate a signal indicative of a first physiological parameter of the subject;
a second sensor, said second sensor is one or more sensors positioned within the monitoring device and capable of electrically coupling to the subject to generate a signal indicative of a second physiological parameter of the subject;
a physiological board having a second connector located on the monitoring device to receive the first sensor at the first connector;
a unit housing having at least two apertures adapted to receive the first sensor and the second sensor, wherein the unit housing houses electronic components of the monitoring device and said unit housing is conformable to the subject; and
a microcontroller unit, electrically disposed within the monitoring device and configured to:
acquire a time-varying electrical signal associated with at least one physiological parameter of the subject from said second sensor and said first sensor electrically attached to the monitoring device via said first connector respectively;
process the electrical signal by quantifying said signal into one or more sets of physiologic data based, at least in part, on a plurality of measurable signals based on the first and on the second physiological parameters from said first sensor and said second sensor to determine one or more physiological conditions of the subject; and
monitor at least one physiological condition associated with the subject;
wherein the microcontroller unit further identifies whether to request an external data from said first sensor.

2. The monitoring unit of claim 1, wherein at least one of the at least two apertures comprises a conductive covering to reduce interference from an external light source and said conductive covering is positioned to provide electrical coupling between the second sensor and the subject.

3. The monitoring unit of claim 1, wherein the unit housing comprises a conductive water resistant or water proof material capable of electrically coupling to the subject and made of synthetic, polymeric or fabric substance to protect the monitoring device from moisture or forces when the first sensor is attached to or detached from the second connector on the monitoring device.

4. The monitoring unit of claim 1, wherein the microcontroller unit is configured and securely adapted to communicate said one or more sets of physiologic data based, at least in part, on the first and on the second physiological parameters from said first sensor and said second sensor with a network.

5. The monitoring unit of claim 1, further comprising a signal conditioning circuitry configured to filter, amplify and isolate the electrical signal to and from said first sensor and said second sensor wherein said circuitry is electrically coupled to the microcontroller unit.

6. The monitoring unit of claim 1, further comprising an interface configured to receive inputs from a user, and display the first and the second physiological parameters of the subject received from said first sensor and said second sensor to a user.

7. The monitoring unit of claim 1, wherein the first sensor includes a motion sensor configured to sense a fetus movement in the belly of the subject.

8. A physiological monitoring method comprising:
electrically attaching, to a monitoring unit, at least one detachable external sensor, via a first connector located on the detachable external sensor to a second connector positioned on the monitoring unit and placing on a subject such a combination;
measuring, by the monitoring unit, a first time-varying electrical signal indicative of a physiological parameter of the subject from said detachable external sensor attached to said monitoring unit;
measuring, by the monitoring unit, a second time-varying electrical signal indicative of a physiological parameter of the subject from a plurality of sensors positioned within said monitoring unit; and
processing, by the monitoring unit, said first time-varying electrical signal and said second time-varying electrical signal by obtaining a first sets of physiologic data based upon, at least in part, on a plurality of said electrical signals and quantifying into one or more sets of physiologic data to determine one or more conditions representing physiological changes of said subject.

9. The physiological monitoring method of claim 8, wherein the subject is selected from the group comprising of human skin, bodily fluid, and atmosphere.

10. The physiological monitoring method of claim 8, wherein the at least one detachable external sensor is a strap or wrap comprising:
an adjustment assembly to fit the monitoring unit when applied to the skin of the subject;
a conductive material that remains in contact with the skin of the subject;
a connector or adaptor assembly configurable to communicate with the monitoring unit;
a pressure applicator to focus pressure on the monitoring unit, so said monitoring unit be in firm electrical contact with said connector or adaptor assembly and said conductive material when applied to the skin of the subject; and
one or more sensors capable of electrically coupling to the subject to generate a signal indicative of a physiological parameter of the subject, wherein said one or more sensors includes a motion sensor configured to sense a fetus movement in the belly of a subject.

11. The physiological monitoring method of claim 10, wherein said strap or wrap is at least in part, made of a stretchable, breathable or pliable material that adheres to the skin of the subject to allow flexibility with body movements when the monitoring unit is placed on said subject.

12. The physiological monitoring method of claim 8, wherein said attaching and said placing step is in communication with a network to receive configurable commands for a particular monitoring mode of operation.

13. The physiological monitoring method of claim 8, further comprising monitoring, by the monitoring unit, said one or more conditions of the subject continuously for a period of time.

14. The physiological monitoring method of claim 8, further comprising providing, by the monitoring unit, a feedback for intervention when the occurrence of an abnormal physiological change of the subject is detected.

15. The physiological monitoring method of claim 8, wherein the processing step comprises communicating, by the monitoring unit, said first time-varying electrical signal, said second time-varying electrical signal, said first sets of physiologic data, said one or more sets of physiologic data and said one or more conditions representing physiological changes of the subject to a network.

16. The physiological monitoring method of claim 8, further comprising displaying, on the monitoring unit, said first time-varying electrical signal, said second time-varying electrical signal, said first sets of physiologic data, said one or more sets of physiologic data and said one or more conditions representing physiological changes of the subject via an interface to provide an indication to a user that an abnormal condition or disease state is present.

17. The physiological monitoring method of claim 16, comprising receiving, on the monitoring unit, one or more inputs from a user by said interface.

18. The physiological monitoring method of claim 8, further comprising suspending, on the monitoring unit, said first time-varying electrical signal, said second time-varying electrical signal, said first sets of physiologic data, said one or more sets of physiologic data and said one or more conditions representing physiological changes of the subject on a readable storage medium.

19. The physiological monitoring method of claim 8, further comprising providing, from a network or cloud system, at least one reward output to a user on the monitoring unit as an indication of healthiness.

20. A method of remote monitoring, the method comprising:
electrically attaching, to a monitoring unit, at least one detachable external sensor, via a first connector located on the detachable external sensor to a second connector positioned on the monitoring unit and placing on a subject such a combination;
measuring, by the monitoring unit, a first time-varying electrical signal indicative of a physiological parameter of the subject from said detachable external sensor attached to said monitoring unit;
measuring, by the monitoring unit, a second time-varying electrical signal indicative of a physiological parameter of the subject from a plurality of sensors positioned within said monitoring unit;
receiving, at a network or cloud system, a plurality of said time-varying electrical signals of a plurality of subjects, from a plurality of monitoring units by a network server;
processing, at the network or cloud system, said plurality of electrical signals in realtime to determine one or more physiological conditions associated with said plurality of subjects by said network server;
storing, at the network or cloud system, said one or more physiological conditions of said plurality of subjects in a database; and
transmitting, from the network or cloud system, said one or more physiological conditions to an external entity involving at least one of a network or cloud system, a plurality of monitoring units, a plurality of base stations or an external device having an operable communication link.

21. The method of claim 20, further comprising displaying, at the base station, at least one or more physiological conditions of said plurality of subjects by an user interface and receiving, at the base station, one or more inputs from a user by said user interface.

22. The method of claim 20, further comprising monitoring, by the network or cloud system, said one or more physiological conditions of said plurality of subjects continuously for a period of time.

23. A system for monitoring physiological parameters of a subject, the system comprising:
a first sensor, said first sensor is at least one detachable external sensor having a first connector to attach to a monitoring device and a one or more sensors capable of electrically coupling to a subject to generate a signal indicative of a first physiological parameter of the subject;
a second sensor, said second sensor is one or more sensors positioned within the monitoring device and capable of electrically coupling to the subject to generate a signal indicative of a second physiological parameter of the subject;
a physiological board having a second connector located on the monitoring device to receive the first sensor at the first connector;
a unit housing having at least two apertures adapted to receive the first sensor and the second sensor, wherein the unit housing houses electronic components of the monitoring device and said unit housing is conformable to the subject;
a microcontroller unit, electrically disposed within the monitoring device and configured to:
acquire a time-varying electrical signal associated with at least one physiological parameter of the subject from said second sensor and said first sensor electrically attached to the monitoring device via said first connector respectively;
process the electrical signal by quantifying said signal into one or more sets of physiologic data based, at least in part, on a plurality of measurable signals based on the first and on the second physiological parameters from said first sensor and said second sensor to determine one or more physiological conditions of the subject; and
monitor at least one physiological condition associated with the subject;
wherein the microcontroller unit further identifies whether to request an external data from said first sensor; and
a transceiver module electrically coupled to the microcontroller unit and located within the monitoring device wherein said transceiver module is configured to transmit said first and said second physiological parameters received from said first sensor and said second sensor respectively, to a base station and a network or cloud system.

24. The system of claim 23, wherein the base station and the network or cloud system are operable to monitor physiological conditions of the subject continuously over a period of time.

25. The system of claim 24, wherein the network or cloud system displays on the base station, a plurality of physiological conditions representing a status of the subject.

26. The system of claim 25, wherein the base station is a device selected from the group consisting of a smartphone, tablet, laptop, camera, watch, workstation, personal digital assistant and computer.
